(19) United States
(12) Patent Application Publication
(10) Pub. No.: US 2024/0358269 A1
(43) Pub. Date: Oct. 31, 2024

(54) METHOD AND APPARATUS FOR PATIENT-SPECIFIC B1 FIELD SHIMMING IN MAGNETIC RESONANCE IMAGING SYSTEMS

(71) Applicant: CANON MEDICAL SYSTEMS CORPORATION, Tochigi (JP)

(72) Inventors: Andrew James Wheaton, Shaker Heights, OH (US); Wayne R. Dannels, Mentor, OH (US)

(73) Assignee: CANON MEDICAL SYSTEMS CORPORATION, Tochigi (JP)

(21) Appl. No.: 18/309,210

(22) Filed: Apr. 28, 2023

(51) Int. Cl. A61B 5/055; A61B 5/00

(52) U.S. Cl. CPC A61B 5/055; A61B 5/7267; A61B 5/0042

(57) ABSTRACT

A method for performing patient-specific B1 field shimming in a magnetic resonance imaging system includes obtaining patient information of a patient to be imaged by the magnetic resonance imaging system. The method further includes determining an orientation of a projection based on the obtained patient information. The method also includes acquiring B1 projection data, using the magnetic resonance imaging system, along the determined orientation of the projection. In addition, the method includes determining a set of B1 shimming parameters based on the acquired B1 projection data.

### FIELD

This disclosure relates to patient-specific shimming of radio frequency (RF) magnetic fields (i.e., “B1 fields”) in magnetic resonance imaging (MRI) systems. The B1 field shimming is performed in vivo based on projection data acquired along one or more projections over a patient that are determined in accordance with the anatomy of the patient to be imaged.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

As part of the procedure for producing MRI images within the body of a patient, a static magnetic field (B0) is used by an MRI scanner to align the nuclear spins of atoms. During the scan, RF pulses generated by an RF transmitter cause perturbations to the local magnetic field, and RF signals emitted by the nuclear spins are detected by an RF receiver.

In order to achieve diagnostic images with high spatial resolution and high contrast resolution, the strength of B0 fields is increasingly higher (from 1.5T to 3T and above) in clinical practice. Under higher B0 fields, however, RF behavior in the patient becomes more complex. For example, the dielectric properties of the human body can cause local perturbations to the B1 fields, resulting in non-uniform excitation. This can introduce errors in the contrast of resultant diagnostic images, potentially leading to misdiagnosis.

To tackle this problem, a common approach is to map the RF transmit magnetic field and perform shimming correction in accordance with the map, so as to obtain a more uniform B1 field distribution. In a spatially resolved B1 map, each pixel represents a measurement of the transmit magnetic field B1 at that location. Besides B1 shimming, the map can also be used for RF transmit calibration (for accurate RF pulse flip angles), parallel transmit (pTx) RF pulse control (pTx is generally necessary for 7T MRIs, for example), and correction of quantitative relaxometry maps (commonly associated with longitudinal relaxation time (T1) mapping). A B1 map can be acquired as a pre-scan procedure, and then the B1 map can be used for calibration, design, or correction of data in subsequent sequence acquisitions during the protocol.

For the sake of saving scan time, it is beneficial for pre-scans to be as short as reasonably possible. Although there exist a number of B1 mapping approaches, a common downside is that the measurement time is impractically prolonged. A B1 shimming method that can be performed to correct the B1 field variation at clinical field strengths (e.g., 1.5T, 3T, etc.) within scan time on the order of one second has yet to be established.

Therefore, it is desirable to address these and other deficiencies of current approaches.

### SUMMARY

The present disclosure relates to a method for performing patient-specific B1 field shimming in a magnetic resonance imaging system. The method includes obtaining patient information of a patient to be imaged by the magnetic resonance imaging system. The method also includes determining an orientation of a projection based on the obtained patient information. In addition, the method includes acquiring B1 projection data, using the magnetic resonance imaging system, along the determined orientation of the projection. Further, the method includes determining a set of B1 shimming parameters based on the acquired B1 projection data.

The disclosure additionally relates to an apparatus for performing patient-specific B1 field shimming in a magnetic resonance imaging system. The apparatus comprises processing circuitry that obtains patient information of a patient to be imaged by the magnetic resonance imaging system. The processing circuitry also determines an orientation of a projection based on the obtained patient information. In addition, the processing circuitry acquires B1 projection data, using the magnetic resonance imaging system, along the determined orientation of the projection. Further, the processing circuitry determines a set of B1 shimming parameters based on the acquired B1 projection data.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the disclosure and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIGS. 1(a) and 1(b) show exemplary dielectric artifacts in MRI images;

FIGS. 2(a)-(d) show exemplary B1 field maps acquired in four different imaging anatomies, i.e., head, abdomen, breasts, and thighs;

FIG. 3 shows a non-limiting example of a block diagram of a B1 field shimming apparatus according to one embodiment of the present disclosure;

FIG. 4 shows a non-limiting example of a flow chart of a B1 field shimming method according to one embodiment of the present disclosure;

FIG. 5 shows a non-limiting example of a block diagram of projection determination circuitry that determines one or more projections along which projection data is acquired by the MRI system, according to one embodiment of the present disclosure;

FIG. 6 shows a non-limiting example of a flow chart of a process of determining one or more projections along which projection data is acquired by the MRI system, according to one embodiment of the present disclosure;

FIGS. 7(a)-(f) show a non-limiting example of different projections and projection data acquired along the projections respectively, according to one embodiment of the present disclosure;

FIGS. 8(a)-(c) show a non-limiting example of different projection combinations identified for different imaging anatomy, according to one embodiment of the present disclosure;

FIG. 9 shows a non-limiting example of a block diagram of shimming parameter resolving circuitry that resolves a set of shimming parameters based on projection data acquired, according to one embodiment of the present disclosure;

FIG. 10 shows a non-limiting example of a flow chart of a process that resolves a set of shimming parameters based on projection data acquired, according to one embodiment of the present disclosure;

FIG. 11 shows a non-limiting example of a block diagram of shimming parameter resolving circuitry that resolves a set of shimming parameters based on projection data acquired, according to one embodiment of the present disclosure;

FIG. 12 shows a non-limiting example of a flow chart of a process that resolves a set of shimming parameters based on projection data acquired, according to one embodiment of the present disclosure;

FIGS. 13(a)-(d) show a non-limiting example of acquiring projection profiles along two or more orientations, with respect to different sets of shimming parameters, according to one embodiment of the present disclosure;

FIGS. 14(a)-(d) show a non-limiting example of acquiring projection profiles along one or more orientations, with respect to different sets of shimming parameters, according to one embodiment of the present disclosure;

FIG. 15 shows a non-limiting example of a block diagram of shimming parameter resolving circuitry that resolves a set of shimming parameters based on projection data acquired, according to one embodiment of the present disclosure;

FIG. 16 shows a non-limiting example of a flow chart of a process that resolves a set of shimming parameters based on projection data acquired, according to one embodiment of the present disclosure; and

FIG. 17 is a schematic block diagram of a magnetic resonance imaging system, according to one embodiment of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting.

For example, the order of discussion of the different steps as described herein has been presented for clarity's sake. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present disclosure can be embodied and viewed in many different ways.

Furthermore, as used herein, the words “a,” “an” and the like generally carry a meaning of “one or more,” unless stated otherwise.

When electromagnetic waves encounter an object in an MRI scanner, dielectric effects occur: the wavelength decreases, electrical current is generated, and wave reflection/refraction develops at tissue interfaces. Under a high B0 field (3T and above), the homogeneity of the time-varying RF magnetic field (B1) will be highly dependent on the electrical properties of the object, e.g., the body of a patient. As standing wave currents flow in opposite directions from two sides of the patient, a pattern with destructive interference (dark areas) and constructive interference (bright areas) separated by on the order of quarter wavelengths is created. These abnormal bright and dark areas due to B1 field inhomogeneity are commonly labeled as “dielectric artifacts.”

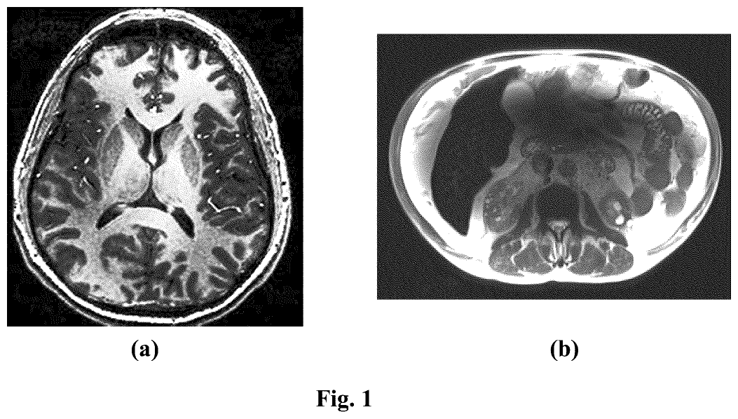

FIGS. 1(a) and 1(b) show exemplary dielectric artifacts in MRI images of human bodies. As can be seen in FIG. 1(a), the center and anterior of the brain image is abnormality brightened. In FIG. 1(b), the center of the abdomen image is abnormality darkened. These dielectric artifacts increase the difficulty of analyzing the MRI images and obtaining clinical information, and in severe cases, can lead to misdiagnosis.

To mitigate the dielectric artifacts, one approach is to acquire information representing the B1 field distribution for each patient such that B1 shimming can be conducted for each patient. This can improve homogeneity of the B1 field, and thus improve image quality and possibly the specific absorption rate (SAR) or B1 rms safety.

Many B1 shimming methods have been developed. Typically, these methods include acquiring a spatially resolved map using standard Cartesian phase encoding. Because B1 is slowly spatially varying, the encoding matrix can be small (corresponding to a low resolution). Commonly, the matrix sizes are 32×32, 48×48, or 64×64, for example. A single slice acquired at the isocenter is often used as a representation of the B1 distribution of the volume. At the cost of increased scan time, 3D B1 maps can also be acquired.

However, even with small matrix sizes (e.g., 32×32, 48×48, or 64×64), it takes several tens of seconds or even a few minutes to acquire data sufficient to build a B1 map. Even though certain fast methods (e.g., the Bloch-Siegert Shift method) can be used, it still requires 64 shots (32 phase encodes×2 frequency offsets) to create a low-resolution B1 map. For example, at TR=200 ms, it would take 12.8 seconds to acquired B1 data for each channel. When separate B1 maps are acquired for each of two channels, the total pre-scan time is about 26 seconds.

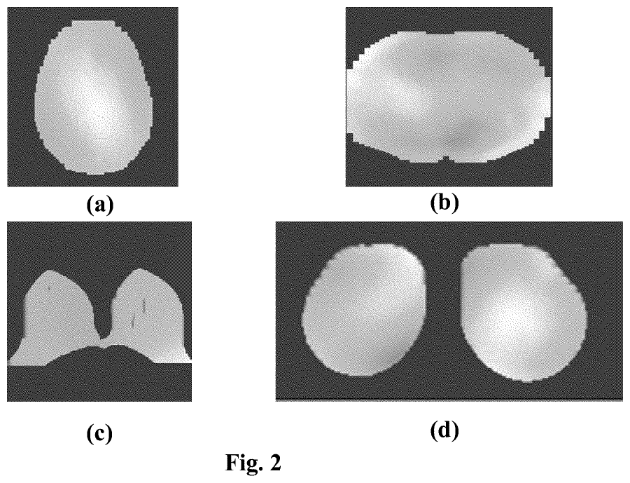

In the context of typical clinical B1 shimming, a fully Cartesian-encoded map is not necessary. Typically, there are only two control parameters: attenuation and phase offset {A, φ}(i.e., settings for the TX2 channel relative to the TX1 channel, perhaps for two drive ports on a quadrature TX coil). Moreover, it is known that the B1 field is slowly spatially varying with a characteristic+/−pattern due to the dielectric effects. In addition, a rough estimate of the shape of the B1 field is known. FIGS. 2 (a)-(d) show exemplary B1 field maps acquired for four different imaging anatomies, i.e., head, abdomen, breasts, and thighs. Each of the B1 maps illustrated in FIGS. 2(a)-(d) has a characteristic distribution, which corresponds to a +/−pattern caused by the dielectric effects. Individual patient maps fit similar characteristic patterns, differing in aspect ratio, spatial scaling, scaling of B1 nonuniformity, etc.

Therefore, when used for purposes of B1 shimming, a B1 map essentially contains no more useful information than a 2×2 image. By acquiring projection data instead of Cartesian encoding, sufficient information can be extracted from one or more 1D projections. In particular, the symmetry of the projection data can reveal adequate information for calculating the optimal {A, φ} set. A map is not necessary in order to solve for {A, φ}; what is required is a few metrics of the B1 spatial distribution. In this way, by acquiring only a few (1, 2, 3, or 4, for example.) projections instead of 32 phase encodes, the scan time can be significantly reduced from tens of seconds to approximately one second.

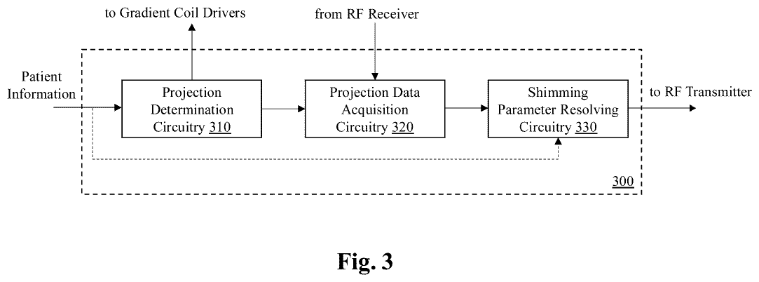

FIG. 3 shows a non-limiting example of a block diagram of a B1 field shimming apparatus according to one embodiment of the present disclosure. The B1 field shimming apparatus 300 includes projection determination circuitry 310, projection data acquisition circuitry 320, and shimming parameter resolving circuitry 330. The projection determination circuitry 310 obtains information regarding the patient to be imaged, determines one or more projections along which projection data is to be acquired, determines gradient signals that are required to achieve the projections, and applies the gradient signals to gradient coil drivers of the MRI scanner. The projection data acquisition circuitry 320 acquires projection data from the RF receiver of the MRI scanner, and sends the projection data to the shimming parameter resolving circuitry 330. Based on the projection data (and the optional patient information), the shimming parameter resolving circuitry 330 resolves and outputs a set of shimming parameters to the RF transmitter of the MRI scanner.

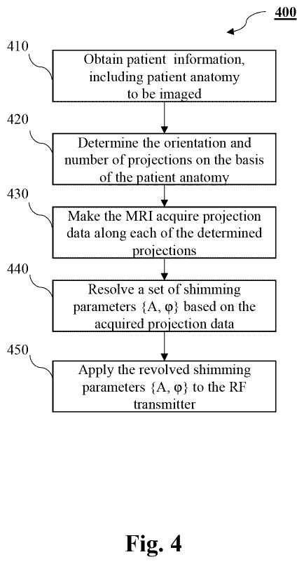

FIG. 4 shows a non-limiting example of a flow chart of a B1 field shimming method according to one embodiment of the present disclosure. The process 400 of the B1 field shimming method begins at step 410 by obtaining patient information. The patient information includes patient anatomy to be imaged. At step 420, the orientation and number of projections are determined based on the imaging anatomy. The projection orientations may initially be specified relative to patient anatomical axes, such as left-right, head-foot, and anterior-posterior. Among the patient information, there may be information about the orientation of the patient within the MRI-scanner, such as prone-versus-supine, and head-first positioning or feet-first positioning. The anatomical projection orientations plus the patient orientation are sufficient to define the projections in the scanner hardware coordinates, such as x, y and z gradient directions. This kind of geometric transformation is well known in MRI. At step 430, the MRI scanner is caused to acquire projection data along each of the determined projections. At step 440, a set of shimming parameters are resolved based on the acquired projection data. Optionally, the patient information is also used in resolving of the shimming parameters. At step 450, the resolved shimming parameters are applied to the RF transmitter to control individual transmit channels of the MRI scanner.

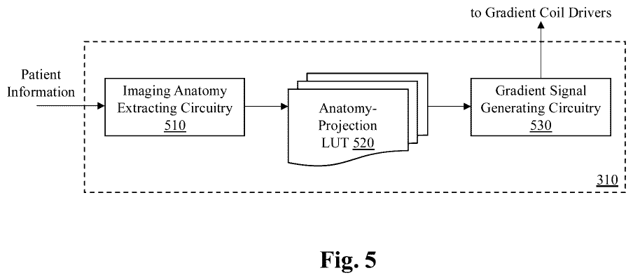

The components of FIG. 3 and the processes performed by those components will be described in more detail below with reference to FIGS. 5, 6, 9, 10, 11, 12, 15, and 16.

FIG. 5 shows a non-limiting example of a block diagram of projection determination circuitry that determines one or more projections along which projection data is acquired by the MRI scanner, according to one embodiment of the present disclosure. The projection determination circuitry 310 includes imaging anatomy extracting circuitry 510, anatomy-projection look-up table 520, and gradient signal generating circuitry 530. The imaging anatomy extracting circuitry 510 receives information regarding the patient and extracts the patient anatomy to be imaged. Many anatomy-projection pairs are prestored in the anatomy-projection look-up table 520. By comparing the imaging anatomy against the entries stored in the look-up table 520, one or more matched projections are searched out and sent to the gradient signal generating circuitry 530. Upon reception of the matched projections, the gradient signal generating circuitry 530 generates gradient signals that are required to achieve the projections and outputs them to the gradient coil drivers of the MRI scanner.

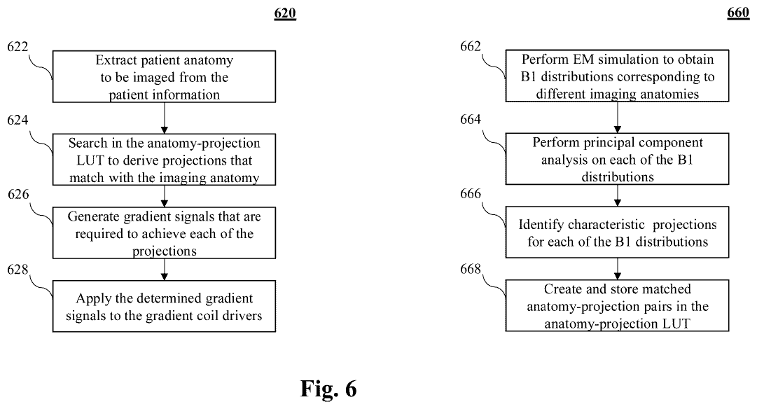

FIG. 6 shows a non-limiting example of a flow chart of a process of determining one or more projections along which projection data is acquired by the MRI scanner, according to one embodiment of the present disclosure. On the left side is a process 620 for determining the projections in accordance with the patient information received. At step 622, information regarding the imaging anatomy is extracted from the patient information. At step 624, a search is performed in the anatomy-projection look-up table 520 to extract one or more projections that match with the imaging anatomy. At step 626, gradient signals required to achieve each of the projections are generated. At step 628, the gradient signals are applied to the gradient coil drivers of the MRI scanner.

On the right side of FIG. 6 is a process 660 of offline preparing the anatomy-projection look-up table 520 used in the process 620. At step 662, electromagnetic simulations are performed to obtain B1 distributions corresponding to different imaging anatomies. At step 664, principal component analysis (PCA) is performed on each of the B1 distributions. At step 666, one or more characteristic projections are identified for each of the B1 distributions. At step 668, matched anatomy-projection pairs are created and stored in the anatomy-projection look-up table 520. Although electromagnetic simulations are used in FIG. 6 to prepare the anatomy-projection look-up table 520, one skilled in the art can appreciate that other means are possible. For example, data for creating the anatomy-projection pairs can be collected from other physical simulations, research experiments on phantoms or volunteers, and/or clinical procedures on patients.

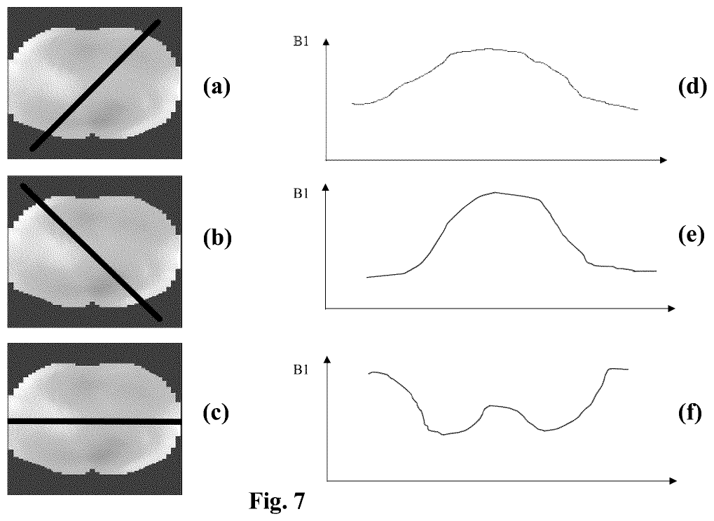

FIGS. 7(a)-(f) show a non-limiting example of different projections and B1 projection data acquired along the projections respectively, according to one embodiment of the present disclosure.

Typically, for B1 shimming, the goal is to solve for two fixed parameters, i.e., {A, φ}. This process is a low-resolution approach for a low degree-of-freedom solution. B1 projection data along a chosen orientation presents a characteristic signal in the projection data and contains enough spatially resolved information about the B1 pattern. When information about the scale of B1 and its spatial distribution is known, B1 shimming parameters {A, φ} can be calculated using the B1 projection data.

In selecting shim parameters, i.e., {A, φ}, various metrics or objectives can be considered. One natural objective is to obtain the shim parameters which produce the least RF amplitude non-uniformity over a slice, as an integral over the slice. Another criterion could be to minimize the peak-to-peak variation over the slice. Yet other criteria are possible. Of particular interest may be the asymmetry of the variance in the B1 RF field. The human body has significant left-right symmetry, for example, in the brain. This symmetry can assist physicians in detecting various clinical conditions. A medical condition may cause some portion of brain, for example, to appear brighter or darker in an MRI image than the corresponding anatomical region on the contralateral side. Reducing other confounding technical sources of image asymmetry can make the task of diagnosis less complicated. Thus, even if overall image appearance is somewhat non-uniform due to RF transmit field non-uniformity, if that residual nonuniformity largely preserves left-right symmetry, the image may be preferred by clinicians.

In considering RF asymmetry, a single projection in the left-right direction may be judged in how close it comes to being symmetric. Or if two projections are disposed at different angular orientations, but if they correspond to a left-right mirrored pair of lines, then the asymmetry might be judged between the two projections, possibly flipping the values of the RF B1 values end-for-end along one of the two directions.

The selection of the projection orientations can be important to the resolving process. Typically, there exist principal axes for a B1 distribution in human subjects. Using methods such as principal component analysis (PCA), the optimal projection orientations can be identified.

PCA is an algorithm for sorting and comparing elements of variance in data. One exemplary use of PCA for choosing projection orientations is as follows. Collect an ensemble of B1+ map images, covering a range of human subjects, and covering a range of shim parameter settings. Analysis proceeds, so as to emphasize the most significant variance in the population, and/or the aspects of variance which may most successfully modified by applying alternate shim value settings. Reformat the images to have similar centers, sizes, and intensity. Apply a common mask so as to mainly keep pixels which overlap in many of the images. Convert each image into a long 1-dimensional feature vector. Append the 1-dimensional vectors (row-wise or column-wise) into a single rectangular array. Apply the singular value decomposition algorithm (SVD), to identify singular vectors sorted in order of decreasing singular values. The SVD step is similar to an eigenvalue-eigenvector decomposition, but can be performed on rectangular arrays. Extract the highest few 1-dimensional singular vectors. For each, reformat it back from a 1-dimensional feature vector into a 2D image again. Each such resultant image is a principal component. Inspect the first few PCA images for radial directions which show the most overall variance, when the maps are projected along those radial readout directions. These are favorable and sensitive directions for acquiring shimming projections.

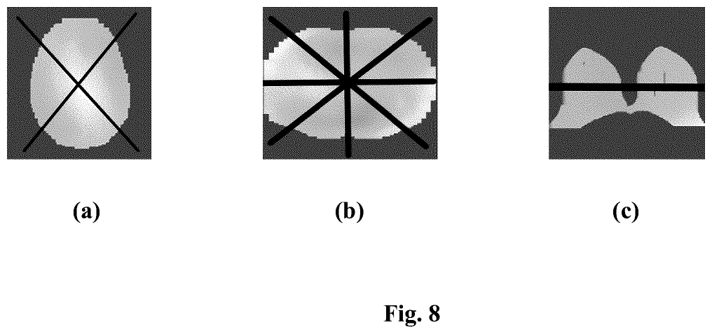

FIGS. 8(a)-(c) show a non-limiting example of different projections identified for different imaging anatomies. Each anatomy presents a characteristic B1 pattern. The number of projections can be specific for each imaging anatomy. A plurality of different models can be chosen to be included for each imaging anatomy, so as to cover “corner cases” (for example, metal implant, amputation/resection, etc.).

The orientation of the projections also can be specific for each imaging anatomy. The orientation can be chosen through PCA to align with the characteristic B1 pattern of the anatomy, for example. As illustrated in FIGS. 8(a)-(c), two orthogonal projections, oriented at about 45° can be chosen for the head (8(a)). For the body (8(b)), 2 orthogonal projections or 4 projections separated by about 45° can be chosen. Both breasts and thighs can use one horizontal projection due to their simple B1 patterns. Note that the specific angles shown in FIGS. 8 (a)-(c) are not restrictive. Other angular orientations are possible without departing from the scope of the present disclosure. For example, an angle of 40°, 60°, etc., can be used instead of 45° shown in FIG. 8(a).

In one non-limiting embodiment, a minimum number of projections necessary for B1 shimming is determined to save scan time. Taking the head as an example, two projections can be determined. When the Bloch-Siegert Shift method is used, only four shots (two projections and two frequency offsets for each projection) are needed to obtain sufficient information. With this projection-based shimming approach, B1 shimming data can be acquired in less than one second. In an alternative embodiment, more projections can be acquired in order to add stability to the solution. As such, the orientation and number of the projections illustrated in FIGS. 8(a)-(c) is not limiting upon the present disclosure.

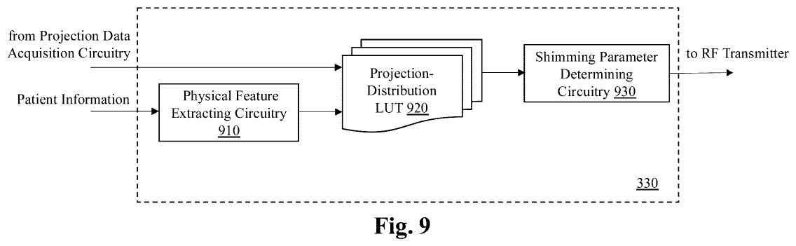

In the following, multiple approaches for solving a final output of {A, φ} from the B1 projection data will be described with reference to FIGS. 9-16.

In one non-limiting embodiment, translation from the B1 projection data to a final output of shimming parameters {A, φ} is conducted by a search within a dictionary or look-up table. It is observed that most patients share a characteristic B1 pattern. For example, it is known that axial slices of heads have a finite range of elliptical aspect ratios, e.g., from circle to ellipse. For a given size and aspect ratio, the patterns are very similar.

Thus, for each imaging anatomy, a group of B1 distributions can be pre-defined for different dimensional scales and aspect ratios, for example. More elements can be added to take into account features in more dimensions, such as body fat composition, male/female, age (e.g., child, youth, adult, senior), etc. Such a group of B1 distributions can be built offline by electromagnetic simulations using Sim4Life or a similar platform. Projection data of each of these B1 distributions can be generated, and a {A, φ} solution for each B1 distribution can be solved a priori. Thus, a look-up table can be prepared, each entry of which includes a B1 distribution and physical features, projection data, and a {A, φ} solution that are correlated to that B1 distribution. Then, a dictionary search (by brute force or other means) can be conducted in the look-up table to find a B1 distribution that has matched B1 projection data and matched physical features. For the matched B1 distribution, the optimized {A, φ} is already known.

FIG. 9 shows a non-limiting example of a block diagram of shimming parameter resolving circuitry that resolves a set of shimming parameters based on closely matching projection data acquired, according to one embodiment of the present disclosure. The shimming parameter resolving circuitry 330 includes physical feature extracting circuitry 910, a projection-distribution look-up table 920, and shimming parameter determining circuitry 930. The physical feature extracting circuitry 910 extracts one or more physical features from the patent information. The physical features can be a dimensional scale and/or an aspect ratio, for example. Based on the projection data received from the projection data acquisition circuitry 320 and the extracted physical features, a matched B1 distribution is searched out in the projection-distribution look-up table 920. Based on the matched B1 distribution, the shimming parameter determining circuitry 930 determines a set of shimming parameters and applies them to the RF transmitter.

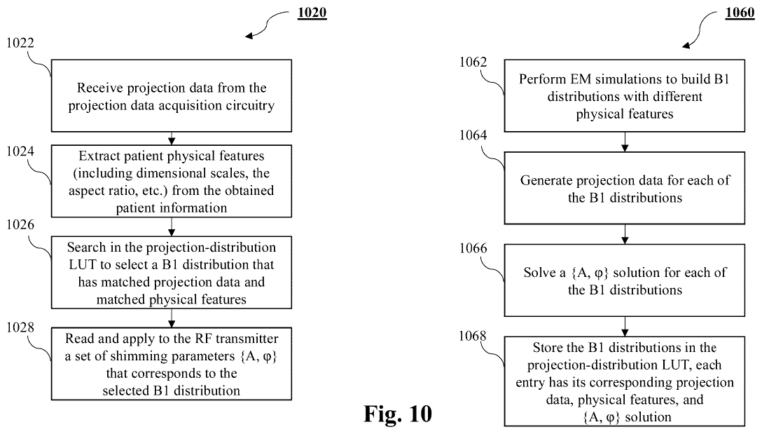

FIG. 10 shows a non-limiting example of a flow chart of a process that resolves a set of shimming parameters based on projection data acquired, according to one embodiment of the present disclosure. On the left of FIG. 10 is a process 1020 for resolving the shimming parameters using the projection-distribution look-up table 920. At step 1022, the projection data is received from the projection data acquisition circuitry 320. At step 1024, one or more physical features (e.g., a dimensional scale and/or an aspect ratio, etc.) are extracted from the patient information. At step 1026, a search is performed in the pre-stored projection-distribution look-up table 920 to extract a B1 distribution that has matched projection data and matched physical features. At step 1028, a set of shimming parameters that are correlated with the derived B1 distribution are read and applied to the RF transmitter of the MRI scanner.

On the right side of FIG. 10 is a process 1060 for offline preparation of the projection-distribution look-up table 920. At step 1062, electromagnetic simulations are performed to build B1 distributions for different physical features (including, but not limited to, a dimensional scale and/or an aspect ratio, etc.). At step 1064, projection data is generated for each of the B1 distributions. At step 1066, a shimming parameter solution is solved for each of the B1 distributions. At step 1068, the B1 distributions are stored in the shimming parameter look-up table 920. Each entry has a B1 distribution and projection data, physical features, and a shimming parameter solution that are correlated to the B1 distribution. Although electromagnetic simulations are used in FIG. 10 to prepare the projection-distribution look-up table 920, one skilled in the art can appreciate other means are possible. For example, data for creating the distribution entries can be collected from other physical simulations, research experiments on phantoms or volunteers, and/or clinical procedures on patients.

In an alternative embodiment, machine learning is used to map one set of information into another domain. In this case, physical features and the measured spatial B1 projection data are inputted into a neural network. The output of the neural network is the optimal shimming parameter set {A, φ}. Instead of looking up the {A, φ} solution in a pre-defined dictionary or look-up table, the machine learning approach learns the mapping with the neural network and supervised learning. The network learns the transfer function in advance from training data including B1 distributions, physical features, projection data, and shimming parameter sets {A, φ}. As it is computationally faster to execute a machine learning inference than a dictionary search, this approach gives an additional advantage in speed.

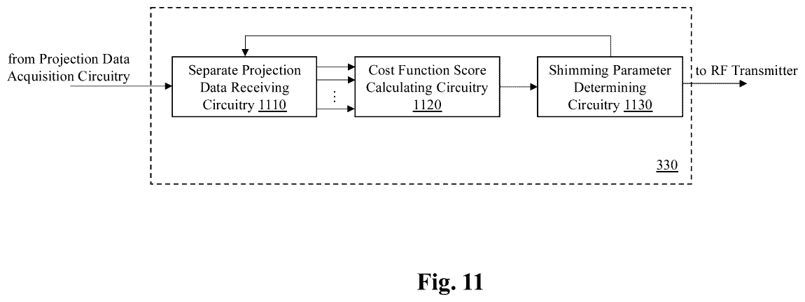

FIG. 11 shows a non-limiting example of a block diagram of shimming parameter resolving circuitry that resolves a set of shimming parameters based on projection data acquired, according to one embodiment of the present disclosure. The shimming parameter resolving circuitry 330 includes separate projection data receiving circuitry 1110, cost function score calculating circuitry 1120, and shimming parameter determining circuitry 1130. The shimming parameter determining circuitry 1130 selects a plurality of different shimming parameter sets applied to the RF transmitter. The separate projection data receiving circuitry 1110 obtains projection data from the projection data acquisition circuitry 320, and outputs to the cost function score calculating circuitry 1120 separate projection data acquired along one or more orientations, with the different shimming parameter sets applied. The cost function score calculating circuitry 1120 calculates a cost function score with respect to each of the plurality of different shimming parameter sets. Based on the cost function scores, the shimming parameter determining circuitry 1130 identifies a set of shimming parameters that has the lowest cost function score, and outputs the set of shimming parameters to the RF transmitter of the MRI scanner.

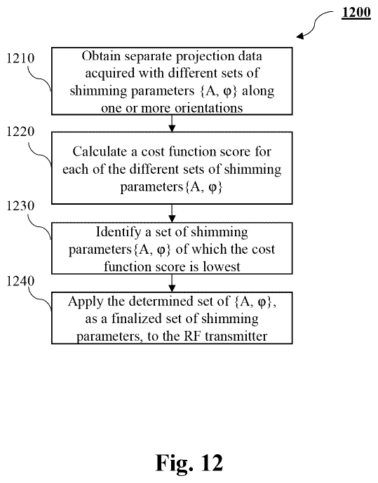

FIG. 12 shows a non-limiting example of a flow chart of a process 1200 that resolves a set of shimming parameters based on projection data acquired, according to one embodiment of the present disclosure. At step 1210, with respect to different sets of shimming parameters, projection data is received along one or more orientations. For example, projection data can be obtained along two orientations (e.g., about 45° and about 135°, about 0° and about 90°, etc.), for different sets of shimming parameters (for example, one parameter set can be {A=0 dB, φ=50° } and another can be {A=3 dB, φ=120° }). At step 1220, a cost function score is calculated for each of the different sets of shimming parameters. As described below, the cost function score can be based on an L1-norm or an L2-norm, for example, and cost function scores in other forms are possible as well. At step 1230, based on the cost function scores calculated in step 1220, a set of shimming parameters is identified that has the lowest cost function score. At step 1240, the identified set of shimming parameters is determined as the finalized set of shimming parameters and applied to the RF transmitter.

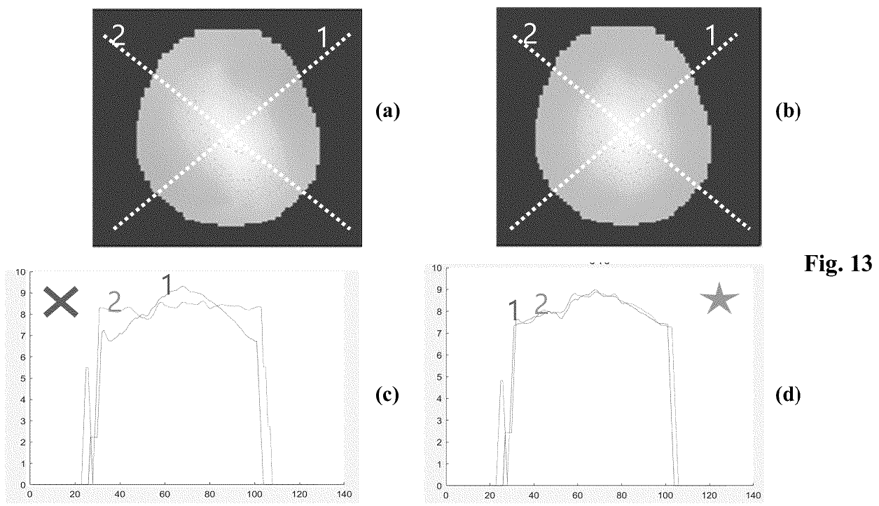

FIGS. 13(a)-(d) show a non-limiting example of acquiring projection profiles along two or more orientations, with respect to different sets of shimming parameters, according to one embodiment of the present disclosure. FIG. 13(a) shows two exemplary orientations, labeled with 1 and 2, with a first set of shimming parameters applied to the RF transmitter. Projection data profiles generated along the two orientations 1 and 2 are shown in FIG. 13(c). Similarly, FIG. 13(b) shows two orientations 1 and 2 with a second set of shimming parameters applied, and the corresponding projection data profiles acquired are shown in FIG. 13(d). It is appreciated that the numbers of the sets of shimming parameters and the orientations are not restrictive. Without departing from the scope of the disclosure, other numbers of shimming parameter sets and orientations are feasible.

According to Equation (1) below, a cost function score is calculated for each set of shimming parameters, so as to measure the difference between the projection data profiles acquired along orientations 1 and 2:

$$Cost function score = \frac{{\sum }_{x = 1}^{x = Nx} {( \vert B {1}_{1} ( x ) - B {1}_{2} ( x ) \vert )}^{L}}{Nx} \qquad ( 1 )$$

wherein L=1 or 2, x denotes an index of pixels along the projection data profile, and Nx refers to the number of all of those pixels. Alternatively, Nx refers to the number of the pixels within a certain summation range. For example, the summation range can be a range for which there is good data, a range for which the signal magnitude is higher than some threshold or some noise floor, or a range that is windowed or cropped to exclude potentially poorer quality data at the ends of the profile curve (e.g., keeping the central 80% of the data while discarding 10% of the values on either end), etc. Given L=1, an L1-norm-based score is obtained from the calculation of Equation (1). The L1-norm is the sum of absolute values of the difference between the projection data profiles along the orientations 1 and 2.

Here, the L1-norm-based score is effectively a measure of symmetry of the B1 field. As the projection profiles shown in FIG. 13(d) have a smaller difference between each other than that between the two projection profiles shown in FIG. 13(c), the L1-norm-based score calculated for the second set of shimming parameters is smaller than that for the first set of shimming parameters. The smaller cost function score reveals that the second set of shimming parameters is better as it leads to a more uniform B1 field distribution. Therefore, by comparing cost function scores calculated for a plurality of different sets of shimming parameters, a best set of shimming parameters can be identified.

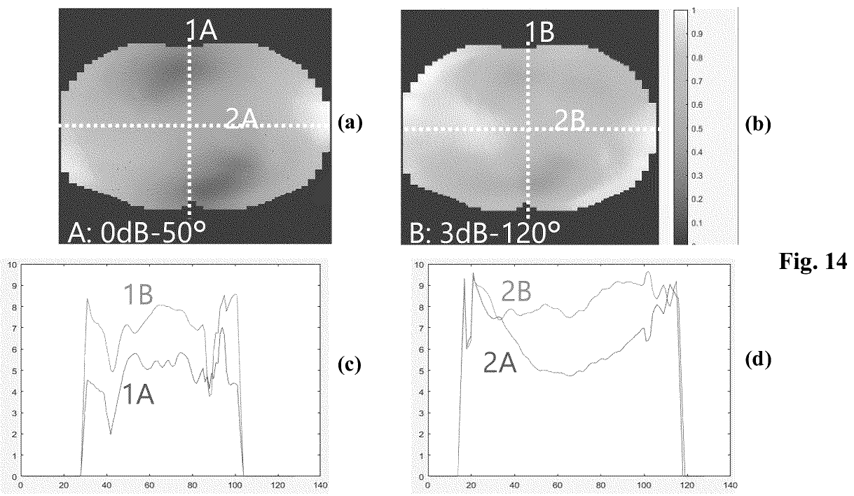

It is possible to evaluate a cost function score in other forms than the L1-norm-based score to identify a best set of shimming parameters. FIGS. 14(a)-(d) show a non-limiting example of acquiring projection profiles along one or more orientations, with respect to different sets of shimming parameters, according to one embodiment of the present disclosure. FIG. 14(a) shows two exemplary orientations, labeled with 1A and 2A, with a first set of shimming parameters (e.g., A=0 dB; φ=50°) applied to the RF transmitter. The corresponding projection data profiles 1A and 2A obtained along the two orientations are shown in FIGS. 14(c) and 14(d), respectively. Similarly, FIG. 14(b) shows two orientations 1B and 2B with a second set of shimming parameters (e.g., A=3 dB; φ=120°) applied, and the corresponding projection data profiles 1B and 2B acquired are shown in FIGS. 14(c) and 14(d).

For each set of shimming parameters, an L2-norm-based cost function score can be calculated according to Equation (2) below:

$$Cost function score = \frac{{\sum }_{x = 1}^{x = Nx} {( \vert B {1}_{1} ( x ) - B {1}_{target} \vert )}^{2}}{Nx} \qquad ( 2 )$$

wherein x and Nx have similar meanings as those in Equation (1), and B1_{target} refers to a preferred B1 projection data profile that can be obtained with any feasible means. The cost function score in this embodiment is based on an L2-norm, which represents the sum of squared differences between the projection data profile along an orientation and fixed, target B1 field data. The projection data profile obtained along either one or both of the two orientations shown in FIGS. 14(a) and (b) can be used in calculation of this L2-norm-based score for the two different sets of shimming parameters. Again, by comparing cost function scores calculated for a plurality of different sets of shimming parameters, a best set of shimming parameters can be identified.

With the approach shown in FIGS. 11 and 12, it is possible to select two projections (e.g., shown in FIGS. 13(a)-(d)) or even only one projection (e.g., shown in FIGS. 14(a)-(d)) to optimize the B1 shimming. Alternatively, more projections can be added to improve the robustness of the solution.

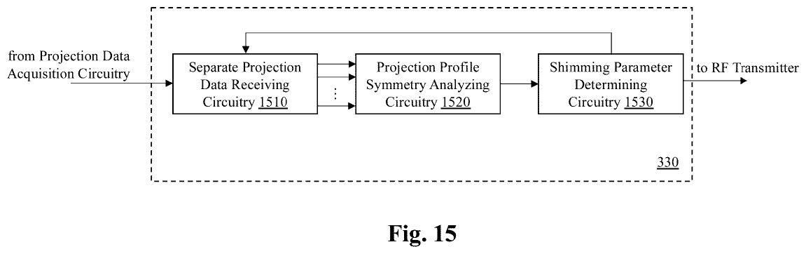

FIG. 15 shows a non-limiting example of a block diagram of shimming parameter resolving circuitry that resolves a set of shimming parameters based on projection data acquired, according to one embodiment of the present disclosure. The shimming parameter resolving circuitry 330 includes separate projection data receiving circuitry 1510, projection profile symmetry analyzing circuitry 1520, and shimming parameter determining circuitry 1530. The shimming parameter determining circuitry 1530 generates shimming parameters to be applied to the RF transmitter. In particular, by applying appropriate shimming parameters, the shimming parameter determining circuitry 1530 can switch one of the transmit channels ON, and the other channels OFF. The projection data separating circuitry 1510 receives the projection data acquired with different transmit channels switched ON. The projection profile symmetry analyzing circuitry 1520 evaluates the effect of different transmit channels on the symmetry of the projection profile. Based on the result of the evaluation, the shimming parameter determining circuitry 1530 determines a finalized set of shimming parameters that maximizes the symmetry of the projection profile.

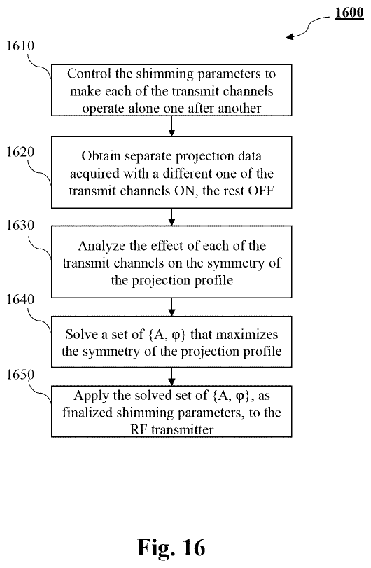

FIG. 16 shows a non-limiting example of a flow chart of a process 1600 that resolves a set of shimming parameters based on projection data acquired, according to one embodiment of the present disclosure. The process starts at step 1610, in which the shimming parameters applied to the RF transmitter are controlled to make each of the transmit channels operate alone. At step 1620, projection data is received from the projection data acquisition circuitry 320. The separate projection data is acquired with a different one of the transmit channels turned ON, the rest OFF. At step 1630, the effect of each of the transmit channels on the symmetry of the projection file is analyzed. At step 1640, a set of shimming parameters {A, φ} is solved. Such a set of shimming parameters maximizes the symmetry of the projection profile. At step 1650, the solved set of shimming parameters {A, φ} is applied, as a finalized set of shimming parameters, to the RF transmitter.

Similar to the approach shown in FIGS. 11 and 12, this approach does not need a pre-calculated look-up table or a trained neural network. Instead, this approach uses the symmetry of the projection profile (or the cost function score calculated for each set of shimming parameters, for the approach shown in FIGS. 11 and 12) as a surrogate measure of uniformity of the B1 field distribution. By evaluating the effect of each transmit channel on the symmetry of the B1 data in the profile, a best set of {A, φ} can be solved to maximize the symmetry.

Preferably, the orientation of the projection is selected along the characteristic directions of the B1 field distribution. More preferably, the orientation of the projection is selected along the characteristic orientation displaying the most asymmetry, so as to add sensitivity to the approach. With this approach, it is possible to select only one projection. Alternatively, more projections can be added to improve the robustness of the solution.

Note that for the approaches illustrated in FIGS. 15, and 16, the choice of projection orientation can be important. If ‘hot’ and ‘cold’ spots (+/−regions) in the B1 field distribution overlap, it will be difficult to differentiate the effect of B1 shimming. Taking the projections shown in FIGS. 7(a)-(c) as an example, 7(a) illustrates a good choice because +/−regions are mostly not overlapped in the projection, i.e., when the B1 amplitudes are combined along a ray perpendicular to the readout direction of the projection. This combination task of forming projections is similar to taking a mean value; the amplitude of the projected data does not increase simply based upon the length of the ray through the subject. Also, 7(b) is a good choice because +/−regions are mostly not overlapped in the projection. However, 7(c) is not a good choice because +/−regions overlap more than 7(a) and 7(b).

For all embodiments described above, any B1 measuring method can be used, including but not limited to, the Double Angle Method (DAM), Actual Flip Angle (AFI), Dual Refocusing Echo Acquisition Mode (DREAM), Phase Sensitive (PS), Saturation Recovery, and the Bloch-Siegert Shift method, for example. The approaches given in this disclosure will perform best with a B1 measuring method immune to tissue composition and relaxation biases. If the B1 data is contaminated by different tissue types, the projection will have an error, because it is effectively an integration of all tissue along the projection. Thus, the Bloch-Siegert Shift method is preferable because it is generally largely unaffected by a wide range of tissue compositions.

Preferably, a region-of-interest within the projection can be chosen. For example, only the central 80% of the projection is considered within the solution so as to avoid subcutaneous fat. Conventional Cartesian B1 mapping methods normally would allow support using a region-of-interest centered anywhere in the volume. Any shape, size, and position of the region-of-interest is possible with a fully encoded map. In contrast, the projection-based approaches described in this disclosure are generally limited in flexibility of selecting region-of-interest locations due to the nature of a projection. B1 intensity information along a perpendicular line is intrinsically combined in the basic one-dimensional readout. Unless further features are implemented in the shim acquisition by projections, there would be no method to collect the data only along some finite subsegment of the perpendicular ray. In other words, data in the spatial dimension orthogonal to the projection readout dimension is effectively averaged when measuring the projection. Therefore, the position in this orthogonal dimension will not be resolved, and thus, the boundaries of the ROI in that dimension will not be resolved.

Further, a portion of the volume can be selected. For example, the inner-volume method with RF slice selection along different gradient axes (e.g., 2D spatial RF selection, a “ZOOMit” or “pencil beam” method) can be used. The pencil beam approach (sometimes called “inner volume selection”) uses two or more RF slice-selective pulses with fully- or partially-orthogonal RF selection gradient directions to invoke an MR signal from only the intersecting geometric regions selected by the RF pulses. It is an effective way to generate a signal only along a rectangular column, after which the readout direction can be applied along the length of the column.

In addition, more than one projection per repetition time (TR) (a “bowtie” method) can be acquired using multiple readouts per excitation. For the “bowtie” readout method, following the RF excitation, the RF pulses and pattern of gradient waveforms in the pulse sequence are designed in such a way as to measure two or more echo signals along different spatial trajectories. Specifically, after the excitation of a slice, a conventional readout may collect data in the k-space along one line at some angle through the center of the k-space. Once that line of data is collected, an extra gradient lobe in some other direction can move the net k-space encoding to an extreme location at some other angular direction relative to the center of the k-space. Then another readout gradient and data sampling can trace back along that second angular direction, through the center of the k-space, and to a geometrically opposite position in the k-space. This configuration, with two or more distinct angular readout directions each intersecting at the center, can be called a “bowtie readout method.”

Compared to conventional Cartesian B1 maps, a much shorter pre-scan time can be achieved with the approaches described in this disclosure. If the Bloch-Siegert Shift method is used and the repetition time (TR) is 200 ms, it takes 0.4 seconds for B1 shimming for breasts or thighs (1 projection×2 frequencies), 0.8 seconds for the head (2 projections×2 frequencies), and 1.6 seconds for the body (4 projections×2 frequencies). Therefore, B1 projection data can be acquired in approximately 1 second, compared to about 26 seconds (32 encodes×2 frequencies×2 channels, if a separate map is measured for each channel) for even the lowest resolution Cartesian B1 map. Moreover, it is inexpensive to select more projections to add more stability to the solution, if necessary. The added costs are on the order of about one more second.

Furthermore, under the same principle as free-breathing radial acquisition, it is expected that breath hold is not necessary for the approaches described in the present disclosure, because they measure simple projections through a center of a volume each time. Also, in general, as the scanning duration of the shimming acquisition projections is greatly reduced, there is a less likely chance of data being inconsistent or corrupted by patient motion. Bulk patient motion in a window of about one second is routinely less significant than the total motion encountered across some tens of seconds.

Although the translation of B1 projection data to the {A, φ} solution requires some computation time, both the look-up-table approach and the machine-learning approach take a limited duration to calculate the {A, φ} solution. This computation time can be hidden during other pre-scans (e.g., a coil map). Traditional B1 shimming solutions require computation time as well to solve individual channels of B1 map input.

In general, the approaches described in this disclosure provide significant scan time savings compared with the conventional methods, and there is no need to acquire a complete B1 map in order to calculate simple two-parameter settings for B1 shimming. By measuring a few projections along characteristic orientations, sufficient information can be obtained from the projection data to determine B1 shimming parameters {A, φ}.

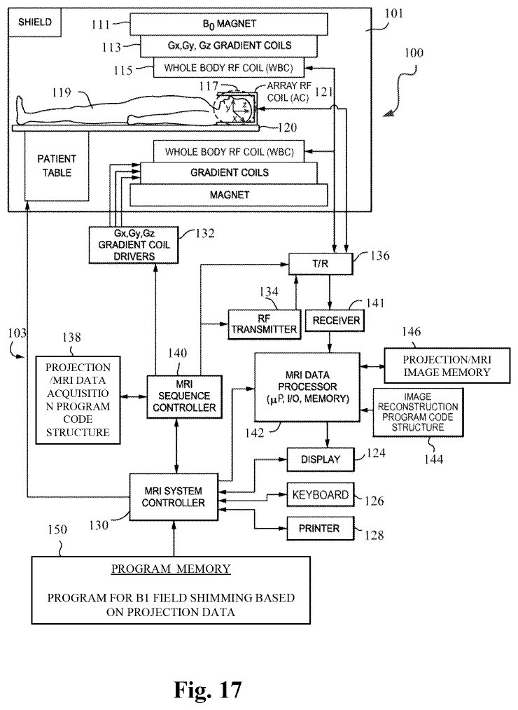

Referring now to FIG. 17, a non-limiting example of a magnetic resonance imaging (MRI) system 100 is shown. The MRI system 100 depicted in FIG. 17 includes a gantry 101 (shown in a schematic cross-section) and various related system components 103 interfaced therewith. At least the gantry 101 is typically located in a shielded room. The MRI system geometry depicted in FIG. 17 includes a substantially coaxial cylindrical arrangement of the static field B0 magnet 111, a Gx, Gy, and Gz gradient coil set 113, and a large whole-body RF coil (WBC) assembly 115. Along a horizontal axis of this cylindrical array of elements is an imaging volume 117 shown as substantially encompassing the head of a patient 119 supported by a patient table 120.

One or more smaller array RF coils 121 can be more closely coupled to the patient's head (referred to herein, for example, as “scanned object” or “object”) in imaging volume 117. As those in the art will appreciate, compared to the WBC (whole-body coil), relatively small coils and/or arrays, such as surface coils or the like, are often customized for particular body parts (e.g., arms, shoulders, elbows, wrists, knees, legs, chest, spine, etc.). Such smaller RF coils are referred to herein as array coils (AC) or phased-array coils (PAC). These can include at least one coil configured to transmit RF signals into the imaging volume, and a plurality of receiver coils configured to receive RF signals from an object, such as the patient's head, in the imaging volume.

The MRI system 100 includes an MRI system controller 130 that has input/output ports connected to a display 124, a keyboard 126, and a printer 128. As will be appreciated, the display 124 can be of the touch-screen variety so that it provides control inputs as well. A mouse or other I/O device(s) can also be provided.

The MRI system controller 130 interfaces with an MRI sequence controller 140, which, in turn, controls the Gx, Gy, and Gz gradient coil drivers 132, as well as the RF transmitter 134, and the transmit/receive switch 136 (if the same RF coil is used for both transmission and reception). The RF transmitter 134 may be composed of two or more transmitter channels for driving two or more RF transmit coils or ports on coils, as is used for RF shimming. The MRI sequence controller 140 includes suitable program code structure 138 for implementing MRI imaging (also known as nuclear magnetic resonance, or NMR, imaging) techniques including B1 field shimming. MRI sequence controller 140 can be configured for MR imaging with or without parallel imaging. Moreover, the MRI sequence controller 140 can facilitate one or more preparation scan (pre-scan) sequences, and a scan sequence to obtain a main scan magnetic resonance (MR) image (referred to as a diagnostic image). MR data from pre-scans can be used, for example, to determine shimming parameters for RF coils 115 and/or 121.

The MRI system components 103 include an RF receiver 141 providing input to data processor 142 so as to create processed image data, which is sent to display 124. The MRI data processor 142 is also configured to access previously generated MR data, images, and/or projection data, such as, for example, projection data acquired with different perturbance in shimming parameters, projection data acquired with different transmit channels, and/or system configuration parameters 146, and program code structures 144 and 150.

In one embodiment, the MRI data processor 142 includes processing circuitry. The processing circuitry can include devices such as an application-specific integrated circuit (ASIC), configurable logic devices (e.g., simple programmable logic devices (SPLDs), complex programmable logic devices (CPLDs), and field programmable gate arrays (FPGAs), and other circuit components that are arranged to perform the functions recited in the present disclosure, such as described with respect to FIGS. 3, 5, 9, 11, and 15.

The processor 142 executes one or more sequences of one or more instructions contained in the program code structures 144 and 150. Alternatively, the instructions can be read from another computer-readable medium, such as a hard disk or a removable media drive. One or more processors in a multi-processing arrangement can also be employed to execute the sequences of instructions contained in the program code structures 144 and 150. In alternative embodiments, hard-wired circuitry can be used in place of or in combination with software instructions. Thus, the disclosed embodiments are not limited to any specific combination of hardware circuitry and software. For example, the program code structure 150 can store instructions that when executed perform method 400.

Additionally, the term “computer-readable medium” as used herein refers to any non-transitory medium that participates in providing instructions to the processor 142 for execution. A computer readable medium can take many forms, including but not limited to, non-volatile media or volatile media. Non-volatile media includes, for example, optical, magnetic disks, and magneto-optical disks, or a removable media drive. Volatile media includes dynamic memory.

Also illustrated in FIG. 17 is a generalized depiction of an MRI system program storage (memory) 150, where stored program code structures such as instructions to perform method 400 are stored in non-transitory computer-readable storage media accessible to the various data processing components of the MRI system 100. As those in the art will appreciate, the program store 150 can be segmented and directly connected, at least in part, to different ones of the system 103 processing computers having most immediate need for such stored program code structures in their normal operation (i.e., rather than being commonly stored and connected directly to the MRI system controller 130).

Additionally, the MRI system 100 as depicted in FIG. 17 can be utilized to practice exemplary embodiments described herein below. The system components can be divided into different logical collections of “boxes” and typically comprise numerous digital signal processors (DSP), microprocessors and special purpose processing circuits (e.g., for fast A/D conversions, fast Fourier transforming, array processing, etc.). Each of those processors is typically a clocked “state machine” wherein the physical data processing circuits progress from one physical state to another upon the occurrence of each clock cycle (or predetermined number of clock cycles).

Furthermore, not only does the physical state of the processing circuits (e.g., CPUs, registers, buffers, arithmetic units, etc.) progressively change from one clock cycle to another during the course of operation, the physical state of associated data storage media (e.g., bit storage sites in magnetic storage media) is transformed from one state to another during operation of such a system. For example, at the conclusion of an image reconstruction process and/or sometimes an image reconstruction map (e.g., coil sensitivity map, unfolding map, ghosting map, a distortion map etc.) generation process, an array of computer-readable accessible data value storage sites in physical storage media will be transformed from some prior state to a new state wherein the physical states at the physical sites of such an array vary between minimum and maximum values to represent real world physical events and conditions. As those in the art will appreciate, such arrays of stored data values represent and also constitute a physical structure, as does a particular structure of computer control program codes that, when sequentially loaded into instruction registers and executed by one or more CPUs of the MRI system 100, causes a particular sequence of operational states to occur and be transitioned through within the MRI system 100.

Numerous modifications and variations are possible in light of the above teachings. It is therefore to be understood that within the scope of the appended claims, the disclosure can be practiced otherwise than as specifically described herein.

Embodiments of the present disclosure may also be as set forth in the following parentheticals.

(1) A method for performing patient-specific B1 field shimming in a magnetic resonance imaging system, comprising: obtaining patient information of a patient to be imaged by the magnetic resonance imaging system; determining an orientation of a projection based on the obtained patient information; acquiring B1 projection data, using the magnetic resonance imaging system, along the determined orientation of the projection; and determining a set of B1 shimming parameters based on the acquired B1 projection data.

(2) The method of (1), further comprising controlling the magnetic resonance imaging system based on the determined set of B1 shimming parameters.

(3) The method of (1), wherein the step of determining the orientation further comprises extracting, from the patient information, a specific imaging anatomy, and obtaining, from a first look-up table, using the extracted imaging anatomy as a key, the orientation of the projection, and the acquisition step further comprises generating gradient signals based on the obtained orientation of the projection, and applying the generated gradient signals to gradient coil drivers of the magnetic resonance imaging system.

(4) The method of (3), further comprising determining B1 field distributions corresponding to different particular imaging anatomies, respectively; performing principal component analysis on each of the determined B1 field distributions; identifying, based on a result of the principal component analysis, a characteristic projection for each of the determined B1 field distributions; and storing the identified characteristic projections and the corresponding imaging anatomies as matched pairs in the first look-up table.

(5) The method of (4), wherein the step of determining the B1 field distributions further comprises determining the B1 field distributions based on data collected in a physics simulation, a phantom experiment, an in vivo experiment, and/or a clinical procedure conducted on a patient.

(6) The method of (3), wherein the step of determining the set of B1 shimming parameters further comprises: extracting, from the patient information, a specific physical feature; obtaining, from a second look-up table, a B1 field distribution, wherein a particular physical feature related to the B1 field distribution matches the extracted physical feature, and particular B1 projection data related to the B1 field distribution matches the acquired B1 projection data; and determining a particular set of B1 shimming parameters related to the obtained B1 field distribution to be the determined set of B1 shimming parameters.

(7) The method of (6), further comprising: determining B1 field distributions for different corresponding physical features, respectively; generating corresponding B1 projection data for each of the determined B1 field distributions; determining a corresponding set of B1 shimming parameters for each of the determined B1 field distributions; and storing the determined B1 field distributions in the second look-up table, wherein each of the determined B1 field distributions is stored in association with the corresponding physical feature, the corresponding B1 projection data, and the corresponding set of B1 shimming parameters.

(8) The method of (6), wherein the extracted physical feature includes at least one of a dimensional scale of the imaging anatomy, an aspect ratio of the imaging anatomy, a body fat composition of the patient, a gender of the patient, and an age of the patient.

(9) The method of (3), wherein the step of determining the set of B1 shimming parameters further comprises: extracting, from the patient information, a specific physical feature; applying the extracted physical feature and the acquired B1 projection data to a trained neural network; and determining the set of B1 shimming parameters from outputs of the neural network.

(10) The method of (9), further comprising: determining B1 field distributions for different corresponding physical features, respectively; generating corresponding B1 projection data for each of the determined B1 field distributions; determining a corresponding set of B1 shimming parameters for each of the determined B1 field distributions; and using the determined B1 field distributions, the corresponding physical features, the corresponding B1 projection data, and the corresponding sets of B1 shimming parameters as training data to train the neural network.

(11) The method of (1), wherein the step of determining the set of B1 shimming parameters further comprises: receiving, along the determined orientation of the projection, B1 projection data corresponding to each of a plurality of sets of B1 shimming parameters, respectively; calculating, based on the received B1 projection data, a cost function score corresponding to each of the plurality of sets of B1 shimming parameters, respectively; identifying a particular set of B1 shimming parameters which corresponds to a lowest cost function score; and determining the identified particular set of B1 shimming parameters to be the determined set of B1 shimming parameters.

(12) The method of (11), wherein the step of determining the orientation of the projection further comprises determining a first orientation of the projection and a second orientation of the projection, the step of receiving the B1 projection data further comprises receiving, along the first orientation of the projection, first B1 projection data corresponding to each of the plurality of sets of B1 shimming parameters, respectively, and receiving, along the second orientation of the projection, second B1 projection data corresponding to each of the plurality of sets of B1 shimming parameters, respectively, and the step of calculating the cost function score further comprises calculating, for each of the plurality of sets of B1 shimming parameters, the cost function score based on the received first B1 projection data with respect to the first orientation of the projection and the received second B1 projection data with respect to the second orientation of the projection.

(13) The method of (11), wherein the step of calculating the cost function score further comprises calculating, for each of the plurality of sets of B1 shimming parameters, the cost function score based on the received B1 projection data and target B1 projection data.

(14) The method of (1), wherein the step of determining the set of B1 shimming parameters further comprises: controlling a set of B1 shimming parameters to switch ON each of individual transmit channels in an RF transmitter of the magnetic resonance imaging system, with other transmit channels switched OFF; receiving particular B1 projection data acquired with each of the individual transmit channels switched ON, respectively; analyzing the received particular B1 projection data to evaluate an effect of each of the individual transmit channels on a symmetry of a profile of the B1 projection data; calculating a particular set of B1 shimming parameters that maximizes the symmetry of the profile of the B1 projection data; and determining the calculated particular set of B1 shimming parameters to be the determined set of B1 shimming parameters.

(15) The method of (14), wherein the step of determining the orientation further comprises determining the orientation such that the profile of the B1 projection data acquired along the determined orientation has minimum symmetry.

(16) The method of (1), wherein the set of B1 shimming parameters includes an RF amplitude modulation and an RF phase modulation to be applied to RF transmit channels of an RF transmitter of the magnetic resonance imaging system.

(17) The method of (1), wherein the acquiring step further comprises acquiring the B1 projection data using a Bloch-Siegert Shift method, a Double Angle method, an Actual Flip Angle method, a Dual Refocusing Echo Acquisition Mode method, a Phase Sensitive method, or a Saturation Recovery method.

(18) The method of (1), wherein the acquiring step further comprises performing 2D spatial selection to select a portion within a volume of the patient along the determined orientation of the projection.

(19) The method of (1), wherein the acquiring step further comprises performing more than one readout per excitation to acquire more than one projection per repetition time.

(20) An apparatus for performing patient-specific B1 field shimming in a magnetic resonance imaging system, the apparatus comprising: processing circuitry configured to obtain patient information of a patient to be imaged by the magnetic resonance imaging system; determine an orientation of a projection based on the obtained patient information; acquire B1 projection data, using the magnetic resonance imaging system, along the determined orientation of the projection; and determine a set of B1 shimming parameters based on the acquired B1 projection data.

Those skilled in the art will also understand that there can be many variations made to the operations of the techniques explained above while still achieving the same objectives of the disclosure. Such variations are intended to be covered by the scope of this disclosure. As such, the foregoing descriptions of embodiments of the disclosure are not intended to be limiting. Rather, any limitations to embodiments of the disclosure are presented in the following claims.

What is claimed is:

1. A method for performing patient-specific B1 field shimming in a magnetic resonance imaging system, comprising:
obtaining patient information of a patient to be imaged by the magnetic resonance imaging system;
determining an orientation of a projection based on the obtained patient information;
acquiring B1 projection data, using the magnetic resonance imaging system, along the determined orientation of the projection; and
determining a set of B1 shimming parameters based on the acquired B1 projection data.

2. The method of claim 1, further comprising:
controlling the magnetic resonance imaging system based on the determined set of B1 shimming parameters.

3. The method of claim 1, wherein the step of determining the orientation further comprises:
extracting, from the patient information, a specific imaging anatomy, and
obtaining, from a first look-up table, using the extracted imaging anatomy as a key, the orientation of the projection, and
the acquiring step further comprises:
generating gradient signals based on the obtained orientation of the projection, and
applying the generated gradient signals to gradient coil drivers of the magnetic resonance imaging system.

4. The method of claim 3, further comprising:
determining B1 field distributions corresponding to different particular imaging anatomies, respectively;
performing principal component analysis on each of the determined B1 field distributions;
identifying, based on a result of the principal component analysis, a characteristic projection for each of the determined B1 field distributions; and
storing the identified characteristic projections and the corresponding imaging anatomies as matched pairs in the first look-up table.

5. The method of claim 4, wherein the step of determining the B1 field distributions further comprises determining the B1 field distributions based on data collected in a physics simulation, a phantom experiment, an in vivo experiment, and/or a clinical procedure conducted on a patient.

6. The method of claim 3, wherein the step of determining the set of B1 shimming parameters further comprises:
extracting, from the patient information, a specific physical feature;
obtaining, from a second look-up table, a B1 field distribution, wherein a particular physical feature related to the B1 field distribution matches the extracted physical feature, and particular projection data related to the B1 field distribution matches the acquired projection data; and
determining a particular set of B1 shimming parameters related to the obtained B1 field distribution to be the determined set of B1 shimming parameters.

7. The method of claim 6, further comprising:
determining B1 field distributions for different corresponding physical features, respectively;
generating corresponding projection data for each of the determined B1 field distributions;
determining a corresponding set of B1 shimming parameters for each of the determined B1 field distributions; and
storing the determined B1 field distributions in the second look-up table, wherein each of the determined B1 field distributions is stored in association with the corresponding physical feature, the corresponding projection data, and the corresponding set of B1 shimming parameters.

8. The method of claim 6, wherein the extracted physical feature includes at least one of a dimensional scale of the imaging anatomy, an aspect ratio of the imaging anatomy, a body fat composition of the patient, a gender of the patient, and an age of the patient.

9. The method of claim 3, wherein the step of determining the set of B1 shimming parameters further comprises:
extracting, from the patient information, a specific physical feature;
applying the extracted physical feature and the acquired projection data to a trained neural network; and
determining the set of B1 shimming parameters from outputs of the neural network.

10. The method of claim 9, further comprising:
determining B1 field distributions for different corresponding physical features, respectively;
generating corresponding projection data for each of the determined B1 field distributions;
determining a corresponding set of B1 shimming parameters for each of the determined B1 field distributions; and
using the determined B1 field distributions, the corresponding physical features, the corresponding projection data, and the corresponding sets of B1 shimming parameters as training data to train the neural network.

11. The method of claim 1, wherein the step of determining the set of B1 shimming parameters further comprises:
receiving, along the determined orientation of the projection, B1 projection data corresponding to each of a plurality of sets of B1 shimming parameters, respectively;
calculating, based on the received B1 projection data, a cost function score corresponding to each of the plurality of sets of B1 shimming parameters, respectively;
identifying a particular set of B1 shimming parameters which corresponds to a lowest cost function score; and
determining the identified particular set of B1 shimming parameters to be the determined set of B1 shimming parameters.

12. The method of claim 11, wherein the step of determining the orientation of the projection further comprises determining a first orientation of the projection and a second orientation of the projection,
the step of receiving the B1 projection data further comprises:
receiving, along the first orientation of the projection, first B1 projection data corresponding to each of the plurality of sets of B1 shimming parameters, respectively, and
receiving, along the second orientation of the projection, second B1 projection data corresponding to each of the plurality of sets of B1 shimming parameters, respectively, and
the step of calculating the cost function score further comprises:
calculating, for each of the plurality of sets of B1 shimming parameters, the cost function score based on the received first B1 projection data with respect to the first orientation of the projection and the received second B1 projection data with respect to the second orientation of the projection.

13. The method of claim 11, wherein the step of calculating the cost function score further comprises calculating, for each of the plurality of sets of B1 shimming parameters, the cost function score based on the received B1 projection data and target B1 projection data.

14. The method of claim 1, wherein the step of determining the set of B1 shimming parameters further comprises:
controlling a set of B1 shimming parameters to switch ON each of individual transmit channels in an RF transmitter of the magnetic resonance imaging system, with other transmit channels switched OFF;
receiving particular B1 projection data acquired with each of the individual transmit channels switched ON, respectively;
analyzing the received particular B1 projection data to evaluate an effect of each of the individual transmit channels on a symmetry of a profile of the B1 projection data;
calculating a particular set of B1 shimming parameters that maximizes the symmetry of the profile of the B1 projection data; and
determining the calculated particular set of B1 shimming parameters to be the determined set of B1 shimming parameters.

15. The method of claim 14, wherein the step of determining the orientation further comprises determining the orientation such that the profile of the B1 projection data acquired along the determined orientation has minimum symmetry.

16. The method of claim 1, wherein the set of B1 shimming parameters includes an RF amplitude modulation and an RF phase modulation to be applied to RF transmit channels of an RF transmitter of the magnetic resonance imaging system.

17. The method of claim 1, wherein the acquiring step further comprises acquiring the projection data using a Bloch-Siegert Shift method, a Double Angle method, an Actual Flip Angle method, a Dual Refocusing Echo Acquisition Mode method, a Phase Sensitive method, or a Saturation Recovery method.

18. The method of claim 1, wherein the acquiring step further comprises performing 2D spatial selection to select a portion within a volume of the patient along the determined orientation of the projection.

19. The method of claim 1, wherein the acquiring step further comprises performing more than one readout per excitation to acquire more than one projection per repetition time.

20. An apparatus for performing patient-specific B1 field shimming in a magnetic resonance imaging system, the apparatus comprising:
processing circuitry configured to
obtain patient information of a patient to be imaged by the magnetic resonance imaging system;
determine an orientation of a projection based on the obtained patient information;
acquire B1 projection data, using the magnetic resonance imaging system, along the determined orientation of the projection; and
determine a set of B1 shimming parameters based on the acquired B1 projection data.
